(19) United States
(12) Patent Application Publication
(10) Pub. No.: US 2007/0148041 A1
(43) Pub. Date: Jun. 28, 2007

(54) SYSTEMS METHODS AND KITS FOR PREPARING SPECIMEN SLIDES

(75) Inventors: Barry Hunt, Andover, MA (US); Peter Doward, Harvard, MA (US); Steven Scampini, Groton, MA (US); Patrick Guiney, Concord, MA (US); Peter Albany, Groton, MA (US)

Correspondence Address: VISTA IP LAW GROUP LLP, 12930 Saratoga Avenue, Suite D-2, Saratoga, CA, 95070, US

(73) Assignee: CYTYC CORPORATION, Marlborough, MA (US)

(21) Appl. No.: 11/613,169

(22) Filed: Dec. 19, 2006

Related U.S. Application Data
(60) Provisional application No. 60/753,479, filed on Dec. 22, 2005.
(60) Provisional application No. 60/755,593, filed on Dec. 30, 2005.

(51) Int. Cl. G01N 33/00

(52) U.S. Cl. 422/63; 422/102

(57) ABSTRACT

A system for preparing biological specimen slides comprises a slide preparation apparatus for preparing the specimen slides; a processor configured to control the slide preparation apparatus; and a data retrieval device coupled to the processor and configured to retrieve information from a storage device associated with the slide preparation apparatus or a component used by the slide preparation apparatus, wherein the processor selectively enables operation of the slide preparation apparatus based on the retrieved information.

### CROSS-REFERENCE TO RELATED APPLICATIONS

This application claims the benefit under 35 U.S.C. 119 to U.S. Provisional Application Ser. Nos. 60/753,479, filed on Dec. 23, 2005, and 60/755,593, filed on Dec. 30, 2005 both of which are incorporated herein by reference.

### FIELD OF THE INVENTION

The present invention relates to slide preparation systems and, more particularly, to selectively operating a slide preparation apparatus.

### BACKGROUND

Slide preparation systems that are used to prepare cytological specimens are known. A sample collected from a patient is typically stored in a vial, which is sent to a laboratory where the slide preparation system is located. At the laboratory, the vial is placed in the slide preparation system, which processes the sample to create a slide. In some cases, depending on the type of slide preparation apparatus used, a filter assembly may be used to collect cells of the sample and transfer the cellular material to the slide. After the filter is used to prepare a slide, the filter is typically discarded.

Consumable or disposable components such as filter assemblies, vials and slides that are used by the slide preparation system are manufactured in accordance with the specification of the slide preparation apparatus to ensure that produce the expected results when used with the slide preparation apparatus. Using equipment manufactured according to manufacturer specifications ensures that the specimen is properly prepared and allows a cytotechnologist or physician to make the appropriate diagnosis.

However, unauthorized vendors manufacture and/or sell counterfeit, copycat or replacement components that may be similar, but not identical to, components prescribed by the manufacturer of the slide preparation system. In some cases, purchasers may mistakenly buy these other products based on the mistaken belief that they are made or approved by the slide preparation system manufacturer. In other cases, purchasers may deliberately buy these other products to reduce costs. In both cases, however, using consumable or disposable components that are not approved by or that do not meet manufacturer specifications may produce inferior specimen slides, which may lead to misdiagnosis.

Another issue that may arise is re-use of the same component, e.g., to process slides of different patients. Re-use of medical components raises similar diagnosis and reliability and contamination concerns. In order to prevent reuse of disposable products generally, it is known to use a numbering system in order to uniquely identify products made or approved by a manufacturer. For example, a product manufacturer may use unique serial numbers known as ascension identification numbers, which are incrementally increasing numbers that uniquely identify each product. However, known systems that uniquely identify consumable or disposable products have had limited effectiveness. For example, numeric identifiers and checksums can be imitated by counterfeiters so that it can be difficult to distinguish original manufacturer products and copycat products.

For these reasons, it is desirable to provide an improved system and method that can verify the authenticity of consumable slide preparation system components to ensure that the components are made by a manufacturer of a slide preparation system or an approved vendor. Such systems and methods should also ensure that the products have not been used previously and prevent against deliberate use of unauthorized products.

### SUMMARY

According to one embodiment, a system for preparing a specimen slide having cells of a biological specimen includes a slide preparation apparatus, a processor and a data retrieval device. The slide preparation apparatus is configured for preparing specimen slides, and the processor is configured to control the slide preparation apparatus. The data retrieval device is coupled to the processor and configured to retrieve information from a storage device that is associated with the slide preparation apparatus or associated with a component that is used by the slide preparation apparatus. The processor selectively enables operation of the slide preparation apparatus based on the information retrieved by the data retrieval device.

Another embodiment is directed to a method of preparing a specimen slide having cells of a biological specimen using a slide preparation apparatus. The method includes retrieving information associated with a slide preparation apparatus or a component that is used by a slide preparation apparatus to prepare a specimen slide and selectively operating the slide preparation apparatus based on the retrieved information.

In various embodiments, the processor can be a part of the slide preparation apparatus, and the data retrieval device can be coupled to the processor through a connector. The storage device may or may not be attached to the component. Further, the storage device can be a card, and the component can be a filter assembly for collecting cells or particles of a biological specimen. The data retrieval device can have read/write capabilities to allow the information in the storage device to be changed, e.g., in response to the slide processing apparatus using the component to prepare a specimen slide. The processor can prevent the component from being used by the slide preparation apparatus, e.g., by disabling the slide preparation apparatus, based on the information.

Additionally, in embodiments, multiple slide preparation apparatus can be connected via a network so that information regarding a component used by one slide preparation apparatus can be transmitted to another slide preparation apparatus over the network. Further, in embodiments, information from a storage device associated with the slide preparation apparatus or a component used by the slide preparation apparatus may involve a number components that can be used with the slide preparation apparatus, a number of slides that a slide preparation apparatus is allowed to prepare or an identifier, such as a filter identification number.

In a further alternative embodiment, specimen preparation kit for use in preparing a specimen slide having cells of a biological specimen includes a filter assembly and a storage device. The filter assembly is used to collect particles of a biological specimen, and the storage device is associated with the filter assembly. Information is stored to, and retrieved from, the storage device and provided to a processor that selectively enables operation of a slide preparation apparatus that uses the filter assembly to prepare a specimen slide. The kit can also include a tray that holds the filter and the storage device, which may or may not be attached to the filter assembly and may be a card. Other aspects and features of the embodiments will be evident from reading the following description of the embodiments.

### BRIEF DESCRIPTION OF THE DRAWINGS

Referring now to the drawings in which like reference numbers represent corresponding parts throughout and in which:

FIG. 1 illustrates a system for preparing a specimen slide according to one embodiment;

FIG. 2 is a flow chart of a method of preparing a specimen slide according to one embodiment;

FIG. 3 illustrates a filter assembly that can be used with various embodiments;

FIG. 4 illustrates a specimen preparation kit according to one embodiment that includes a component used to prepare a specimen slide and a card storage device;

FIG. 5 illustrates a system for preparing a specimen slide that includes a card storage device and card reader according to another embodiment;

FIG. 6 illustrates a specimen preparation kit according to another embodiment that includes multiple components and a card storage device;

FIG. 7 is a flow chart illustrating another method of preparing a specimen slide according to another embodiment;

FIG. 8 is a flow chart of a method of preparing a specimen slide and changing information read from a storage device according to one embodiment;

FIG. 9 is a flow chart of a method of preparing a specimen slide depending on a manufacturer identification retrieved from a storage device according to another embodiment;

FIG. 10 is a flow chart of a method of preparing a specimen slide depending on a component identification retrieved from a storage device according to another embodiment;

FIG. 11 is a flow chart of a method of making and marking slide preparation components according to one embodiment;

FIG. 12 illustrates a system for preparing a specimen slide using an identifier and encoded data according to one embodiment;

FIG. 13 is a flow chart of a method of preparing a specimen slide and updating a list of identifiers according to one embodiment;

FIG. 14 illustrates a system having networked slide preparation apparatus that can send and receive identifier information to each other according to one embodiment;

FIG. 15A illustrates an example of a slide preparation apparatus and chamber of a slide preparation apparatus that can be used with various embodiments;

FIG. 15B further illustrates an example of a slide preparation apparatus and chamber of a slide preparation apparatus that can be used with various embodiments;

FIG. 16 illustrates a method of using the slide preparation system of FIGS. 15A-B to prepare a specimen slide;

FIG. 17 illustrates another example of a slide preparation system that can be used with various embodiments; and

FIG. 18 is a diagram of a computer hardware system that can be used to perform various functions and steps in accordance with some embodiments.

### DETAILED DESCRIPTION OF ILLUSRATED EMBODIMENTS

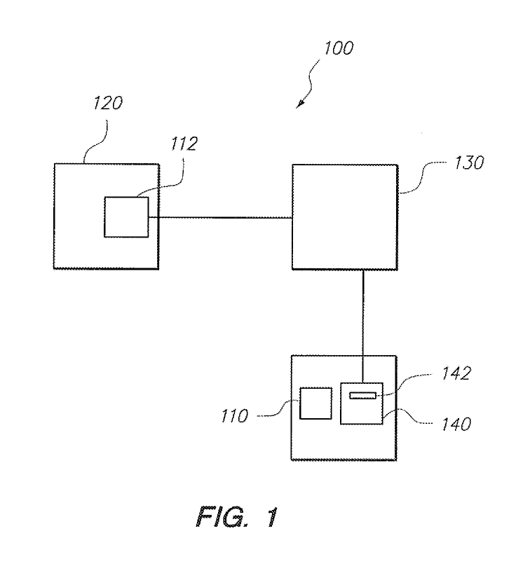

Referring to FIG. 1, one embodiment is directed to a system 100 for selectively operating or controlling a slide preparation apparatus by verifying the authenticity of a component 110 used or consumed by the slide preparation apparatus 120 to prepare a specimen slide. The component 120 may be, for example, a filter assembly, a vial or a slide. The slide preparation apparatus 110 includes or is connected to a processor 112 that is directly or indirectly coupled to a reader or data retrieval device 130. A storage device 140 stores information or data 142. The data retrieval device 130 reads information 142 from the storage device 140 associated with the slide preparation apparatus or a component used by the slide preparation apparatus. The information 142 is provided to the processor 112. The processor is configured, structured or programmed (generally “configured” or “programmed”) to selectively operate or control the slide preparation apparatus 110 according to the information 142 retrieved by the data retrieval device 130.

According to one embodiment, the storage device 140 is a card, e.g., a programmable card, a smart card or a SIM card. For example, a smart card can contain a medium for storing information associated with the filter assembly. In other embodiments, the storage device can be a bar code, a magnetic strip, a diskette, a CD-ROM, an RF device or other suitable storage device. The particular data retrieval device 130 that is used can depend on the particular storage device 140 and the type of information 142 stored on the storage device 140. Further, the information storage device 140 can separate from the component 120 or be affixed to the component 120. For example, the storage device 140 can be a piece of paper (e.g., a sticker or label) having printed or encoded information 142. Thus, the information storage device 140 may be a device that “stores” data in an electronic sense, and may also be other types of medium capable of storing or holding information 142.

The information 142 can be in various forms and may depend on the type of storage device 140 that is utilized. For example, information 142 can be digital data or expressed or represented as a bar code. In such cases, the information storage device 140 (e.g., a sticker or label) may be secured to the component 120, or to another device (e.g., a tag, a card, etc.) associated with the component 120. In further embodiments, the information 142 can be expressed as text and/or numbers, and the data retrieval device 130 can be implemented as a character reader configured to recognize text and/or numbers. Further, in one embodiment, the data retrieval device 130 has write capabilities so that the information 142 can be modified or updated.

Thus, the data retrieval device 130 can be or include a card reader, a camera, a bar code scanner, a magnetic strip reader, a diskette reader, a CD-reader, a RFID transceiver, or other appropriate device to read or retrieve information 142. Further, the reader/writer 130 may also be a card writer, a diskette writer, a CD writer, a magnetic strip writer, a SIM card writer, or other suitable data writing devices. Persons skilled in the art will appreciate that embodiments can be implemented with different storage device 140 data retrieval device 130 configurations having read/write capabilities

In one embodiment in which the storage device 140 is a card, the data retrieval device 130 can be a card reader. During use, the card 140 is inserted into a slot of the card reader 130. As another example, in an embodiment in which the storage device is a barcode, the data retrieval device 130 can be a barcode reader that scans bar code 140 to thereby obtain the information 142 for operation of the slide preparation apparatus 110.

Although FIG. 1 illustrates an embodiment that includes an external data retrieval device 130 connected to a slide preparation apparatus, in alternative embodiments, the data retrieval device 130 can be part of the slide preparation apparatus 110. For example, a component 120 or container or tray that holds or stores one or more components can be inserted into the slide preparation apparatus 110 in order to activate the data retrieval device 130, or the data retrieval device 130 may otherwise be activated by the slide preparation apparatus 110.

The processor 112 can be, for example, an application-specific integrated circuit (ASIC), such as a semi-custom ASIC processor or a programmable ASIC processor. ASICs. Examples of ASICs are described in Application-Specific Integrated Circuits by Michael J. S. Smith, Addison-Wesley Pub Co. (1st Edition, June 1997) and are known in the art of circuit design and, therefore, are not described in further detail. It should be noted that processor 112 can also be any of a variety of circuits or devices that are capable of performing the functions described herein. For example, in alternative embodiments, processor 112 can include a general purpose processor, such as a Pentium processor.

The processor 112 can be a part of the slide preparation apparatus 110 (as illustrated or an external processor 112. For example, the processor 112 can be associated with a computer that is coupled to the slide preparation apparatus 110. For purposes of explanation and illustration, the processor 112 is shown as being a part of the slide preparation apparatus 110, and the data retrieval device 130 is coupled to the processor within the apparatus 110. It should be noted that the processor 112 can have one or multiple processing units or sub-processors.

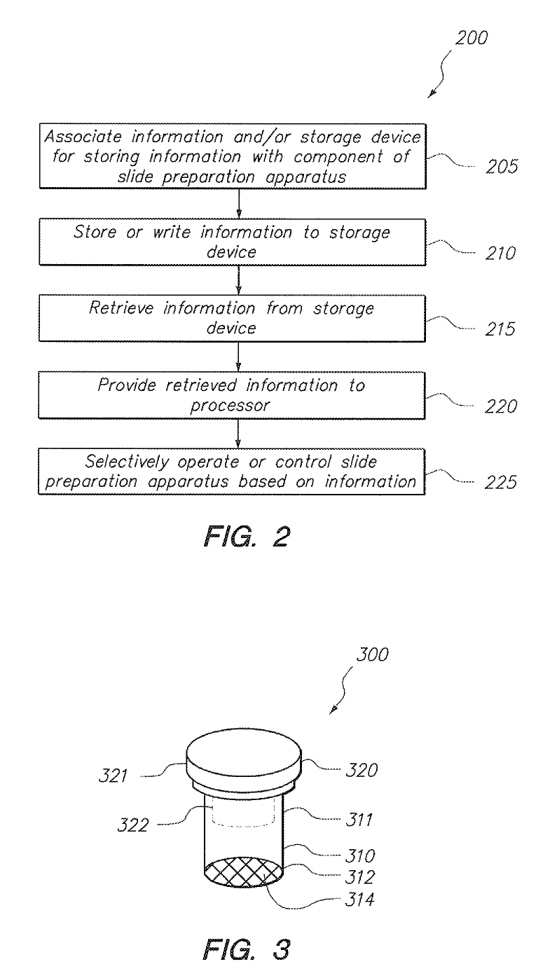

Referring to FIG. 2, a method 200 of preparing a specimen slide according to one embodiment includes associating information and/or a storage device for storing the information with one or more components of a slide preparation apparatus in step 205. In step 210, information from a storage device associated with the slide preparation apparatus or a component used by the slide preparation apparatus is stored or written to the storage device. For example, the storage device can be programmed or encoded with the information. In step 215, the information stored in the storage device is read from or decoded by the reader, e.g., when the component is to be used by the slide processing system to prepare a specimen slide. In step 220, the information is provided by the reader to the processor. In step 225, the processor selectively operates or controls the slide preparation apparatus based on the information.

According to one embodiment, the processor 112 is configured to control whether the component 120 can be used with the slide preparation apparatus 110 to prepare a specimen slide. For example, the processor 112 can control the slide preparation apparatus 110 to prepare a specimen slide using the component based on the information 142. Further, the processor 112 can use the information 142 to select operating parameters of the slide preparation apparatus 110, e.g., parameters for preparing a specimen slide. In another embodiment, the processor 112 can prevent an unauthorized component 120 from being used with the slide preparation apparatus 110. For example, the slide preparation apparatus 110 can be de-activated. Alternatively, the processor 112 can be configured to generate an output or signal that notifies the user that the component 120 should not be used with the slide preparation apparatus 110. The processor can also be configured to initiate other actions as appropriate.

Referring to FIG. 3, according to one embodiment, the component 120 used or consumed by a slide preparation apparatus 110 to prepare a specimen slide is a filter assembly 300. In the illustrated embodiment, a filter assembly 300 includes a filter 310 and a filter cap 320, and the filter 310 has a filter element 314 located at one end 312 of the filter assembly 300. The filter element 314 is used to collect cells or particles from a biological specimen by use of vacuum that draws cell or particle containing fluid up through the filter element 314 so that the cells and particles are collected by the filter element 314.

In the illustrated embodiment, the filter cap 320 has a first portion 321 and a second portion 322. The first portion 321 is sized and shaped to engage with a component of the slide preparation apparatus 110. The second portion 322 is sized and shaped to mate with a first end 311 of the filter 310. Persons skilled in the art will appreciate that embodiments can be applied to other slide preparation components 120. Further, persons skilled in the art will appreciate that the filter assembly 300 shown in FIG. 3 is one example of a filter assembly 300 that can be used with embodiments.

Embodiments can also utilize different types of information 142 from a storage device associated with the slide preparation apparatus or a component used by the slide preparation apparatus. For example, with reference to an embodiment in which the component 120 is a filter assembly 300, information 142 retrieved from a storage device associated with the slide preparation apparatus or a component used by the slide preparation apparatus may include one or more of an identification of a manufacturer of the filter assembly 300, an identification of the filter assembly 300, a value that indicates the number of filter assemblies 300 within a group or kit of filter assemblies 300, a number of slides that can be prepared by the slide preparation apparatus 110, and/or a filter 310 manufactured date. In other embodiments, the information 142 retrieved from a storage device may include data needed during operation of the slide preparation apparatus 110, such as a filter 310 version, calibration information, a processing parameter and other types of information 142 for preparing a specimen slide.

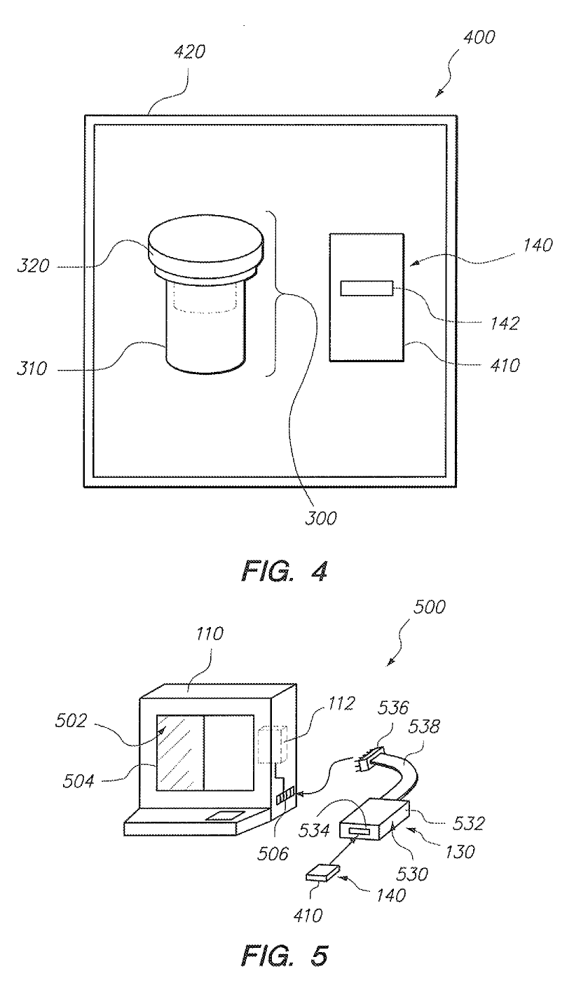

Referring to FIG. 4, according to one embodiment, a kit 400 that is used for preparing a specimen slide includes one or more one or more components 120 and one or more storage devices 140. In the illustrated embodiment, the slide preparation kit 400 includes one filter assembly 300 and one card 410, such as a programmable card, a smart card, a SIM card or other suitable card. In the illustrated embodiment, the filter assembly 300 includes both a filter 310 and a cap 320. In one embodiment, the information 142 stored to the card 410 is related to or associated with the filter assembly 300 and can be removed from the tray 420. Thus, the card 410 is not attached to or embedded within the filter assembly 300 and is independently moveable relative to the filter assembly 300 and the slide preparation apparatus 110. If necessary, the filter assembly 300 and/or other component 120 and the information card 410 or other storage device 140 can be stored in a shipping container, a bag, a box or a tray (as shown in FIG. 4).

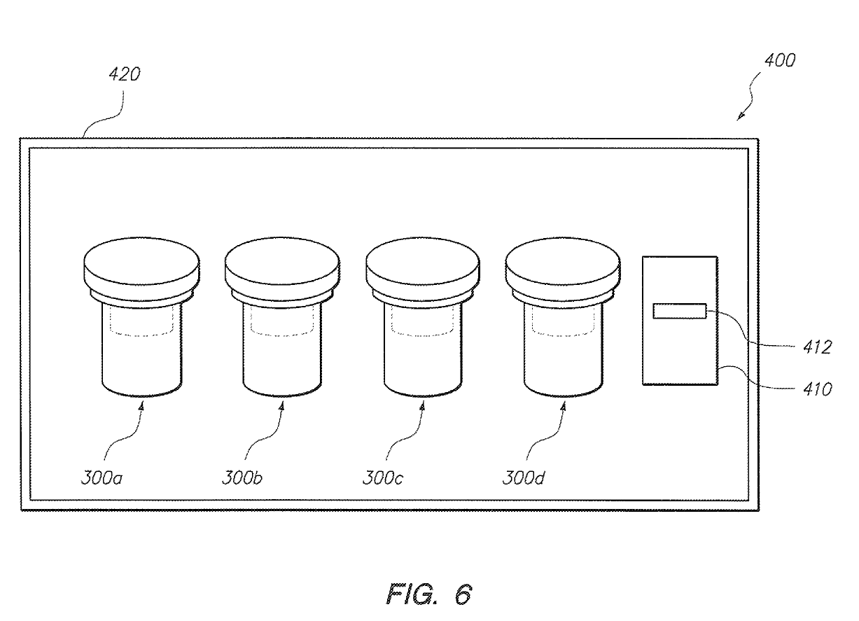

Referring to FIG. 5, in an embodiment in which the storage device is a card 410, e.g., a slide preparation system 500 includes a slide preparation apparatus 110 and a data retrieval device 130 in the form of a card reader 530. In the illustrated embodiment, the slide preparation apparatus 110 has a chamber 502 for housing a filter assembly 300 during use, a door 504 covering the chamber 502, a connector port 506 for electrically connecting another device, such as a data retrieval device 130, to the slide preparation apparatus 110, and a processor 112 coupled to the connector port 506. In the illustrated embodiment, the card reader 530 includes a module or receiving member 532 having a slot 534 for receiving the card 410. The card 410 is removably insertable into the slot 534 formed within the module 532. A cable 538 and a connector 536 connect the reader module 532 to the processor 112 in the slide preparation apparatus 110.

In the illustrated embodiment, the card reader 530 is a separate component and external to the slide preparation apparatus 110. The card reader 530 is connected to the processor 112 in the slide preparation apparatus 110 via a cable 538 and connector 536 during use. In embodiments in which the card reader 530 does not include the cable 538, the module 532 can be coupled directly to the slide preparation apparatus 110. Further, the card reader 530 can be an integrated component of the slide preparation apparatus 110.

FIGS. 4 and 5 illustrate an embodiment in which one card 410 is used, e.g., a card 410 from the specimen preparation kit 400 shown in FIG. 4. In alternative embodiments, a kit 400 can include multiple filter assemblies 300 and/or other components 120. For example, referring to FIG. 6, a specimen preparation kit 400 according to another embodiment includes four filter assemblies 300a-300d (generally 300) and a card storage device 410 for storing information 412 associated with the filter assemblies 300. In the embodiment illustrated in FIG. 6, the specimen preparation kit 400 includes one information card storage device 410 that is associated with multiple filter assemblies 300. In alternative embodiments, a kit 400 can have multiple cards 410.

It should be noted that the specimen preparation kit 400 is not limited to the illustrated configurations, and that the specimen preparation kit 400 can have other components 120, different numbers of components 120, different numbers of storage devices 140, various shapes and sizes, and may or may not be insertable into a slide processing apparatus 110. Further, components 120 of a kit 400 can have different parts. For example, in the illustrated embodiment, the filter assembly 300 includes a filter element 310 and a cap 320. In other embodiments, a specimen preparation kit 400 does not include filter cap(s) 320. In other embodiments, the specimen preparation kit 400 does not include the filter(s) 310. In further embodiments, if the specimen preparation kit 400 includes a plurality of filter assemblies 300, the kit 400 can include a plurality of information storage devices 140 associated with the respective filter assemblies 300. In such cases, each information storage devices 140 may store various types of information such as a number of slides that can be prepared by the apparatus 110 or an identification of the corresponding one of the filter assemblies 300. In any of the embodiments described herein, the specimen preparation kit 400 may be sold by a manufacturer of the kit 400 to a user of the slide preparation system 110.

According to one embodiment, information 142 retrieved from a storage device 140 associated with the slide preparation apparatus or a component used by the slide preparation apparatus is a number or value that indicates how many components 120 are included within a package or kit 400. For example, in embodiments in which the component 120 is a filter assembly 300, the information can be a number 142. Thus, in the specimen preparation kit 400 shown in FIG. 4, the number 142 would be “1” and the number 412 for the specimen preparation kit 400 shown in FIG. 6 would be “4”. Alternatively, a value 412 stored in the card 410 can be a value that is less than the number of filter assemblies 300 in the kit 400. For example, the value may be N−X, wherein N represents the number of filter assemblies 300 in the kit 400, and X represents a factor of safety (which in some cases, can be equal to 0).

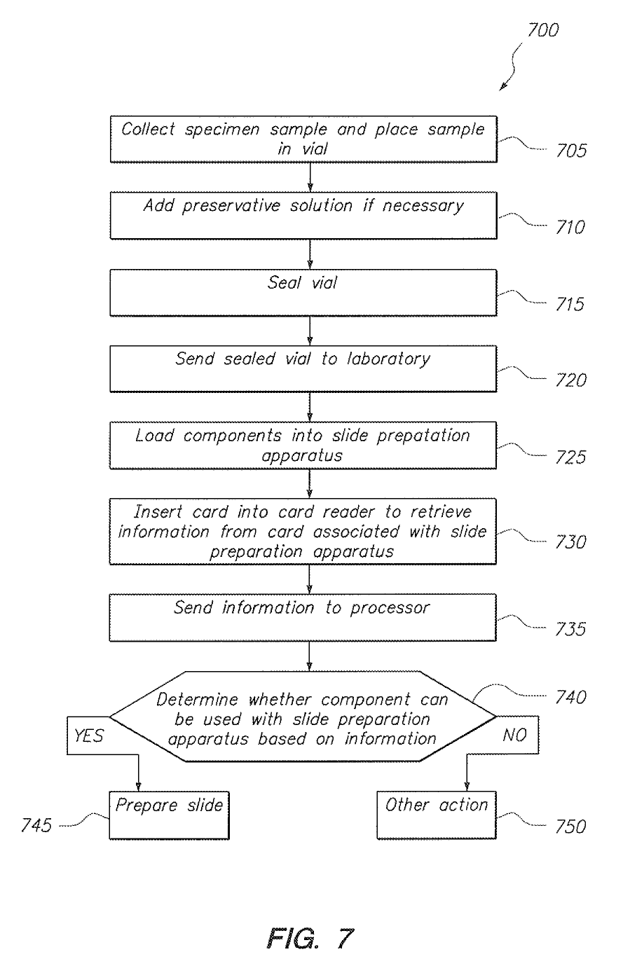

Referring to FIG. 7, a method 700 of using a system and/or specimen preparation kit to according to one embodiment includes collecting a specimen sample and placing the sample in a vial in step 705. If necessary, in step 710, a preservative solution, such as PreservCyt, available from Cytyc Corporation, can be added to the vial. In step 715, the vial is sealed with a cap. In step 720, the sealed vial is sent to a laboratory.

When a specimen slide is to be prepared, a vial, a component, e.g., a filter assembly, and a clean slide are loaded into a chamber of the slide preparation apparatus. In an embodiment in which a specimen preparation kit is utilized, in step 725, a storage device, such as a card can, if necessary, be removed from a tray or container. In step 730, in the embodiment in which the storage device is a card, the card is inserted into the card reader to retrieve information associated with the filter assembly from the storage device. In step 735, the information is sent from the card reader to the processor which, in step 740, determines whether the component can be used with the slide preparation apparatus based on the information. If so, then in step 745, the slide preparation apparatus can use the filter assembly to prepare a slide. If not, then in step 750, the processor can be configured to cause the slide preparation apparatus to take appropriate action. For example, step 750 can involve rejecting the component, de-activating the slide preparation apparatus, generating a signal to the user that the current component is rejected or unauthorized and that another component should be utilized instead or other suitable actions.

According to one embodiment, the processor 112 is configured to operate the slide preparation apparatus 110 to prepare a specimen slide based on information 142 of a value or number indicating a number of components 120 that can be used or a number of specimen slides that a slide preparation apparatus 110 is allowed to prepare. In this embodiment, the slide preparation apparatus 110 may be operable so long as the value 142 is positive. If the value 142 is zero or less than one, then the processor 112 can be configured to prevent the filter assembly 300 from being used with the slide preparation apparatus 110.

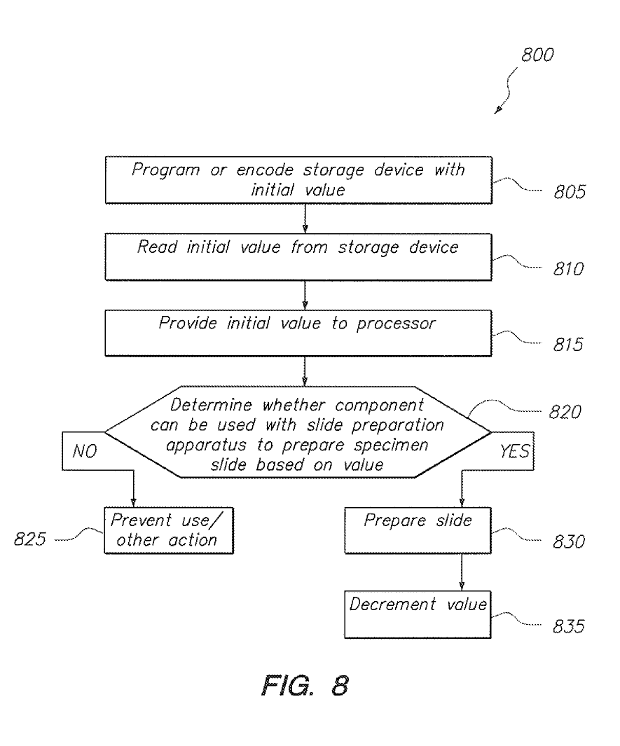

For example, referring to FIG. 8, a method 800 according to one embodiment includes programming or encoding the storage device associated with a component, such as a filter assembly, with an initial value in step 805. In step 810, the initial value is read from the storage device, such as a card, e.g., when the component is to be used by the slide preparation apparatus to prepare a specimen slide. In step 815, the value is transmitted to the processor. In step 820, the processor determines whether the component can be used with the slide preparation apparatus to prepare a specimen slide based on the received value. If not, then in step 825, the processor can be configured to prevent use of the component with the slide preparation apparatus or to take other appropriate action. For example, the processor may be configured to prevent an activation signal from being sent to inhibit the slide preparation apparatus from being activated. In some embodiments, if the processor does not receive data from the reader (e.g., no information storage device is inserted into the slot, or no reader is coupled to the slide preparation apparatus), the processor may be configured to prevent an activation signal from being sent to inhibit the slide preparation apparatus from being activated.

If the component can be used with the slide preparation apparatus, the in step 830, a specimen slide is prepared using the component. In step 835, the value can be decremented to indicate that one less component is available or that one less specimen slide can be prepared. For this purpose, the data retrieval device can have read/write capabilities, and the processor can control the reader to change or update the information stored in the storage device to reflect that one less specimen slide can be prepared.

For example, a specimen kit 400 may include “X” number of filter assemblies 300 and a programmable card 410. After a first specimen slide is prepared, the processor 112 may control the data retrieval device 130 to write or program new value or decrement the original value by 1, i.e., “X−1” to reflect an updated value or data representing, for example, the number of remaining filter assemblies 300 in the specimen kit 400 or the number of specimen slides that can be prepared. The “X−1” value is decremented to “X−2” following preparation of an additional specimen slide, and so on so long as the information 142 indicates that components 120 remain or that specimen slides can be prepared by the slide preparation apparatus 110. Persons skilled in the art will appreciate that although embodiments are described with reference to decrementing values 142, ascending values 142 may also be utilized, e.g., counting up to a given number of components 120 or specimen slides that can be prepared.

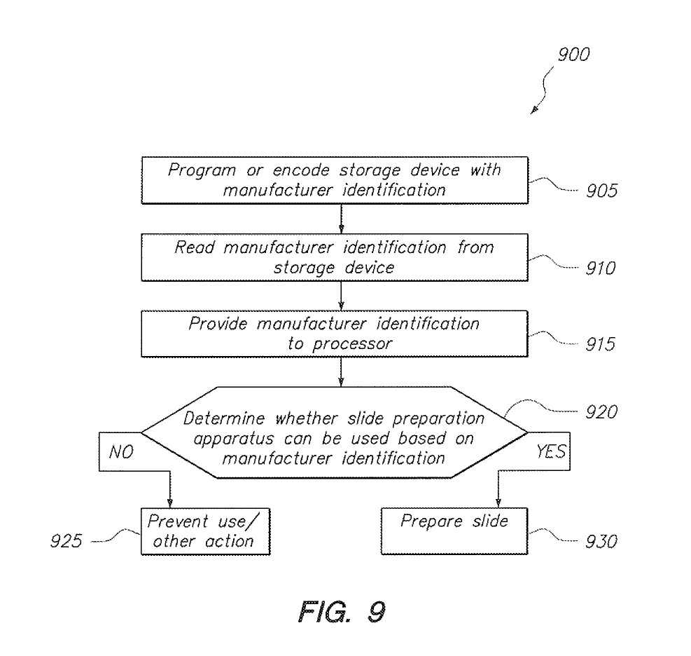

Referring to FIG. 9, in other embodiments, instead of, or in addition to, determining the number of samples that can be processed, the processor 112 can be configured to determine an identification of a manufacturer of the component 120, e.g., a filter assembly 300. A method 900 according to one embodiment includes programming or encoding the storage device with an identification of manufacturer identification in step 905. In step 910, e.g., when the component is to be used to prepare a specimen slide, the manufacturer identification is read from the storage device or card. In step 915, the manufacturer identification read from the storage device is transmitted to the processor. In step 920, the processor determines whether the slide preparation apparatus can be used based on the received manufacturer identification, e.g. by comparing the received manufacturer identification to a list or table of acceptable manufacturer identifications.

If not, then in step 925, the processor can take appropriate action, e.g., preventing the component from being used with the slide preparation apparatus. For example, the processor may be configured so that an activation signal is not sent so that the slide preparation apparatus is disabled. In some embodiments, if the processor does not receive data from the reader (e.g., no information storage device is inserted into the slot, or no reader is coupled to the slide preparation apparatus), the processor may be configured to not send an activation signal to thereby inhibit the slide preparation apparatus from being started. Otherwise, the manufacturer identification received from the reader is acceptable, and in step 930, a specimen slide can be prepared by the slide preparation apparatus using the component.

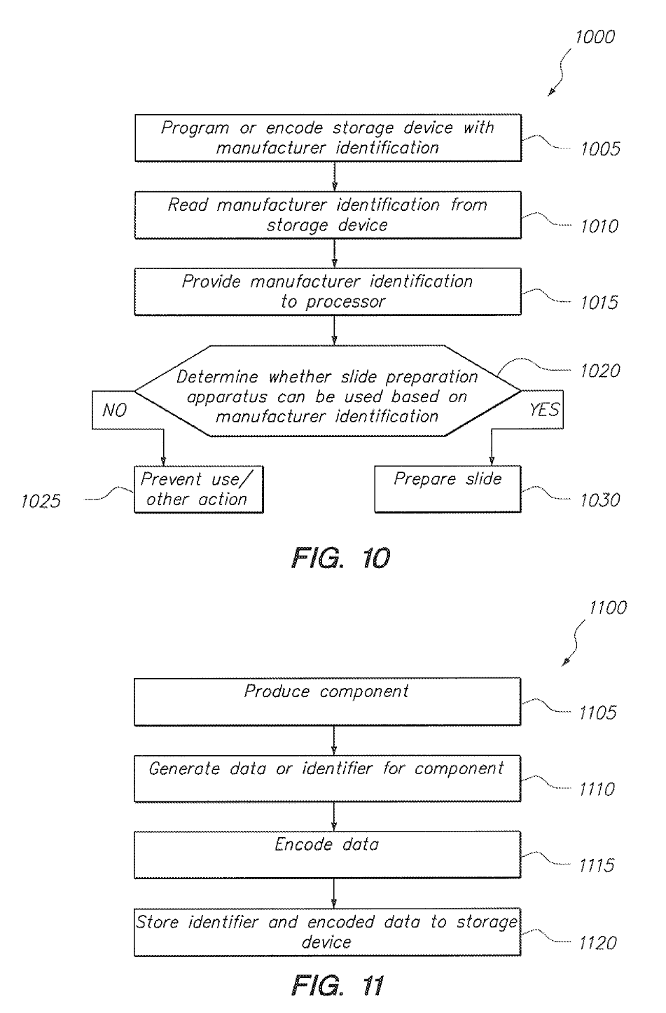

Referring to FIG. 10, in other embodiments, instead of, or in addition to, determining the number of samples that can be processed, the processor 112 can be configured to determine product identification, e.g., a filter assembly 300 identification, by receiving such information 142 from the information storage device 140. A method 1000 according to one embodiment includes programming or encoding the storage device with product identification in step 1005. In step 1010, the product identification is read from the storage device or card associated with a component, e.g., a filter assembly. In step 1015, the product identification is transmitted to the processor. In step 1020, the processor determines whether the component can be used with the slide preparation apparatus can be used based on the received product identification. For example, step 1020 can be performed by comparing the received product identification to a list or table of acceptable product identifications.

If the component is not acceptable, then in step 1025, the processor can take appropriate action such as preventing the component from being used with the slide preparation apparatus. For example, the processor may be configured to not send an activation signal to thereby inhibit the slide preparation apparatus from being activated. In some embodiments, if the processor does not receive data from the reader (e.g., no information storage device is inserted into the slot, or no reader is coupled to the slide preparation apparatus), the processor may be configured to not send an activation signal to thereby disable the slide preparation apparatus from being started. Otherwise, the component can be used with the slide preparation system to prepare a specimen slide in step 1030 if the product identification is acceptable or is a match.

Verifying that the manufacturer of the filter assembly 300 is a an allowable or prescribed manufacturer (e.g., the manufacturer of the slide preparation apparatus, or a manufacturer that has been certified by the slide preparation apparatus 110 manufacturer) or that the filter assembly 300 identification matches a prescribed product identification (e.g., an identification created by a manufacturer of the slide preparation apparatus 110, or by a certified manufacturer of the filter assembly 300) is advantageous in that it ensures that the filter assembly 300 being used with the slide preparation apparatus 110 is compatible with the slide preparation apparatus 110. Further, such verification ensures that components satisfy the required specification associated with an operation of the slide preparation apparatus 110.

Referring to FIG. 11, data or information 142 that is stored to, and retrieved from, the storage device 140, or data or information 142 that is encoded in an identifier attached to or marked upon a component 120, can be encoded. According to one embodiment, a method 1100 of verifying a component such as a consumable or disposable component of a slide processing system includes producing a component in step 1105. In step 1110, data or an identifier is generated for the component. In step 1115, the data or identifier is encoded. In step 1120, the data or identifier and encoded data are stored to a data storage device, such as a smart card or RFID device. The identifier and encoded data from a storage device are associated with the slide preparation apparatus or a component used by the slide preparation apparatus.

Alternatively, the data or identifier and encoded data can be marked onto the component, e.g., by printing the identifier and encoded data onto the component, by printing a barcode containing the identifier and the encoded data onto the component. Thus, in one embodiment, two types of information or data 142 are stored to a storage device, i.e., unencoded data and encoded data. According to one embodiment, a component used with a slide preparation apparatus can include an ascension identifier 1210, and the encoded number or data 1220 may be alpha-numeric numbers printed onto the component, contained in a barcode printed onto the component 120 or other marking or a storage device such as a RFID transponder affixed to the component 120.

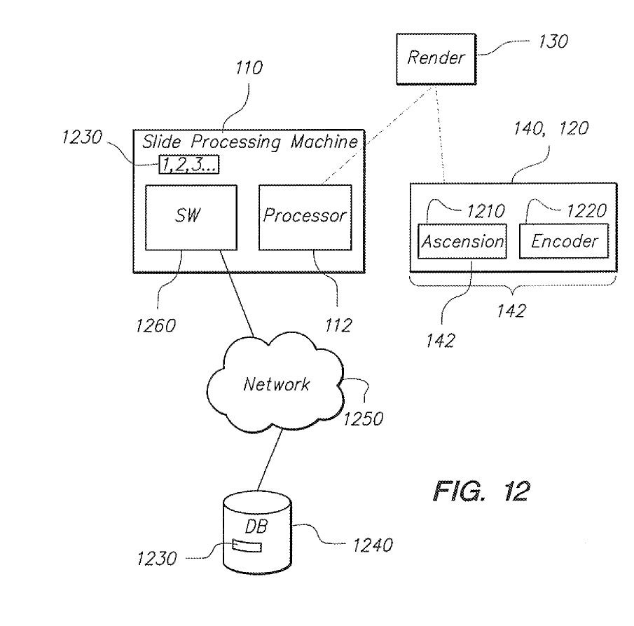

Referring to FIG. 12, in one embodiment, the data or identifier 142 is ascension data or an ascension identifier 1210 (generally ascension identifier) or a unique incrementally increasing identification number. The ascension identifier 1210 can be incremented each time a component 120 is manufactured, thereby ensuring that a unique number is associated with each component 120. Alternatively, other identifiers can be used including other numeric and alpha-numeric identifiers. Thus, an ascension identifier is provided as one example of a component identifier.

According to one embodiment, the information 142 includes encoded data or an encoded number 1220 (generally encoded data). According to one embodiment, encoded data 1220 can be generated based on the ascension identifier 1210. The encoded data 1220 can be generated by an encoding method known to a limited group of authorized users, e.g., authorized vendors and others working in conjunction with authorized vendors. In one implementation, the encoded data 1220 includes CRC or checksum features.

For example, in order to generate the encoded data 1220, a CRC or other checksum can be generated based on the ascension identifier 1210, and the CRC or other checksum could then be encoded 1220 in a manner known to authorized vendors and others working in conjunction with authorized vendors. The encoded CRC or other checksum can then be used as encoded data 1220. As another example, the ascension identifier 1210 can be encoded 1220 in a manner known to authorized vendors and others working in conjunction with authorized vendors, and a CRC or other checksum can be generated based on the encoded ascension identifier 1220. The CRC or other checksum can then be used as the encoded data 1220. Methods for generating CRCs and other checksums are known in the art.

One example of an encoding method that can be used to generate encoded data is applying a known function to the ascension identifier 1210 and then performing a calculating. For example, the ascension identifier 1210 can be multiplied by 3, and added to 24. A CRC of the resulting number can then be generated and used as encoded data 1220.

According to another embodiment, data 1220 is encoded by encryption. For example, encoded data 1220 can be generated using known 128 or 256 bit encryption techniques. In some embodiments, encryption can be used in conjunction with a CRC or other checksum to generate encoded data 1220. According to one embodiment, a unique encrypted identifier 1220 is associated with each package or kit 400 of components 120. The identifier can be encoded 1220 so that it cannot be reproduced or read without an encryption key that is known to the manufacturer or vendor of the slide preparation apparatus 110 and embedded within each processor 112. According to another embodiment, the encrypted identifier 1220 can identify the type of component 120, e.g., a filter assembly 300, a slide, etc., and the lot amount that was originally purchased.

The processor 112 can decrypt or decode the identifier 1220 and determine how many slides can be prepared based on the lot amount. As slides are produced, the stored lot amount value can be adjusted to reflect use of the component 120. For example, if the data retrieval device 130 is a read/write device, the information 142 stored in the storage device 140 can be updated to reflect the number of slides that were prepared and the number of components 120 that were consumed. Further, with embodiments, consumable components 120 can be coupled to particular clients or customers.

The ascension identifier 1210 and encoded data 1220 can be applied directly to a component or marked (e.g., as a barcode) a component 120. Alternatively, the ascension identifier 1210 and encoded data 1220 can be stored to a storage device 140 associated with the component 120. In both cases, a data retrieval device 130 is used to read the information 142 (ascension identifier 1210 and encoded data 1220) from the storage device 140 or directly from the component 120 if the component 120 is marked.

During use, the ascension identifier 1210 and encoded data 1220 are retrieved by a data retrieval device or reader 130 and provided to a processor 112. The processor 112 decodes the encoded data 1220 and determines whether the ascension identifier 1210 and decoded data 1222 satisfy certain criteria to allow the component 120 to be used by the slide preparation apparatus 110 to prepare a specimen slide. The encoded data 1220 can be decoded before or after the ascension identifier 1210 is compared to the criteria.

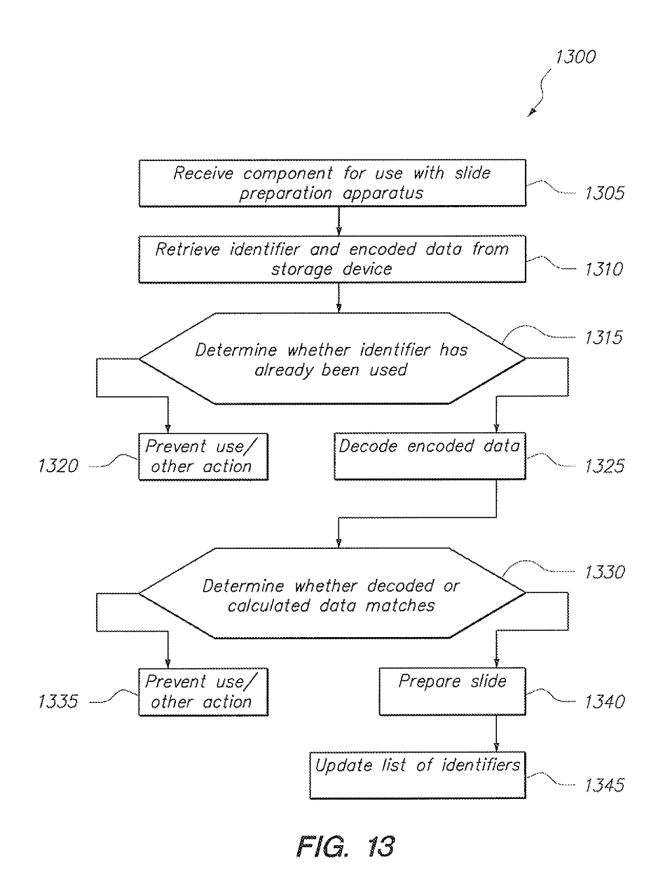

For example, referring to FIGS. 12 and 13, upon receiving a component, such as a filter assembly, for use with a slide processing machine, in step 1305, the ascension identifier and the encoded data are retrieved by a data retrieval device in step 1310. Reading the ascension identifier and the encoded data may involve reading the data directly from a marked component, e.g., a marked filter assembly, or from a storage device associated with a component. In step 1315, a determination is made whether the ascension identifier has already been used, e.g., whether the ascension identifier has already been seen by the processor of the slide processing apparatus. For this purpose, a list or database 1230 of previous ascension identifiers that have been encountered in the past can be locally maintained by the slide processing apparatus. Thus, step 1315 can be performed by a lookup in the database of previous ascension identifiers.

In an alternative embodiment, the slide preparation apparatus may connect or be connected to a remote database 1240 that stores previously seen ascension identifiers 1230 via a network 1250. The remote database 1240 can be maintained, for example, by a vendor of authorized slide processing machines, and may be used to store the ascension identifiers 1210 of all the components, e.g., filter assemblies and slides, that have already been used worldwide.

Determining that the same ascension identifier was seen before may indicate that the component is a counterfeit or a component that does not meet the specifications of the manufacturer of the slide processing machine. For example, some counterfeiters may make multiple copies of a component, duplicating the same ascension identifier on each counterfeit copy. In such cases, in step 1320, the processor can be configured to prevent the component from being used with the slide processing system or issue another suitable output, e.g., a warning to the user.

Otherwise, in step 1325, if the ascension identifier does not match a previously seen identifier, the encoded data can be decoded or calculated, e.g., based on the ascension identifier. In some embodiments, the slide preparation apparatus may be produced by a vendor of the medical equipment, or by a company working in conjunction with the vendor of the machine, and the slide preparation apparatus may be configured to calculate the encoded number using the same encoding scheme that was used by the vendor of the slide preparation apparatus to generate the encoded number.

In step 1330, the processor determines whether the decoded or calculated data, e.g., a number, matches the encoded number that was read from the component. If not, then in step 1335, the processor takes appropriate action such as preventing the component from being sued with the slide preparation apparatus or issuing a warning that the numbers do not match. If the user of the slide preparation apparatus or other medical machinery feels that the component has been rejected in error, for example, due to a mistake in the reading of the ascension identifier or encoded number, the user may reinsert the component into a slide preparation apparatus or cause the verification process to begin.

Numbers that do not match may indicate that the component is a counterfeit or that it does not meet manufacturer specifications. For example, some third party counterfeiters may generate unique ascension identifiers for counterfeit medical equipment. However, because embodiments advantageously generate an encoded number or data in a secret manner, counterfeiters may not be able to generate the appropriate encoded numbers. In this case, the encoded number generated by the counterfeiters and read from the component may not match the encoded number generated by the slide preparation apparatus according to the secret encoding scheme.

Otherwise, in step 1340, the decoded number or data as calculated by the slide preparation apparatus matches the encoded number that was read from the component. In this case, the component may be accepted, and the processor and/or software can permit use of the slide preparation apparatus to prepare a specimen slide.

Further, in step 1345, the list 1230 of ascension identifiers can be updated to include the new ascension identifier associated with the component that is being used by the slide preparation apparatus. In this manner, if the same component is used again, then the slide preparation apparatus will recognize, based on the duplicate ascension identifier, that the component has been previously used.

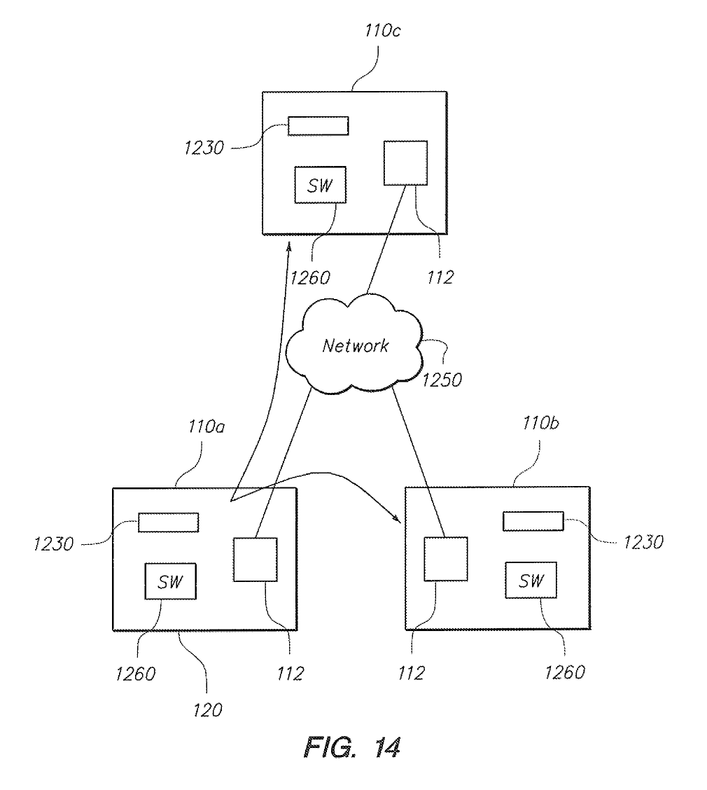

Referring to FIG. 14, in a further alternative embodiment, a slide preparation apparatus 10a can be connected to other slide processing machines 110b and 110c (generally 110) via a network 1250 (such as a Local Area Network or Wide Area Network) so that a component 120 used with one slide preparation apparatus 110 is not utilized with another slide preparation apparatus 110. For example, the slide preparation apparatus 110 can include verification software 1260 that can be used to search ascension identifiers seen by other slide processing machines 110 or to update or send other slide processing machines new ascension identifiers seen by that slide preparation apparatus 110.

The verification software 1260 and/or the processor 112 can be configured to access a database 1240, e.g., via a network 1250, in order to determine whether the ascension identifier 1210 read from the storage device 140 associated with the component 120 has been used before. The verification software 1260 and/or the processor 112 may be configured to compute an encoded number 1220 based on the ascension identifier 1210 read from the storage device 140 associated with the component 120, using an encoding scheme known only to the vendor of the component 120, the manufacturer of the slide preparation apparatus 110 and to others working in conjunction with authorized vendors. The verification software 1260 and/or the processor 112 may also be configured to compare the computed encoded number 1220 to the encoded number read from the component 120.

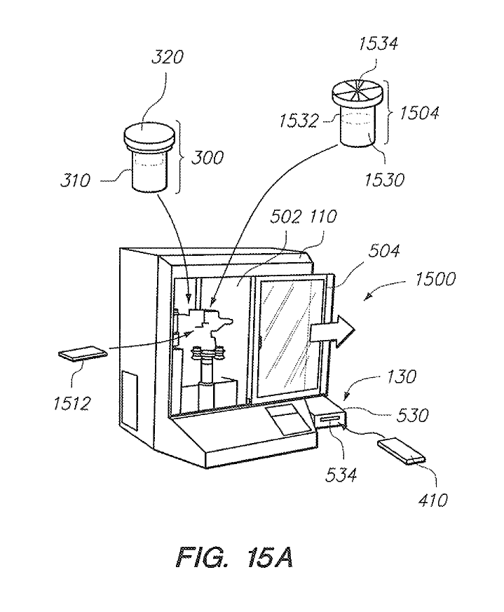

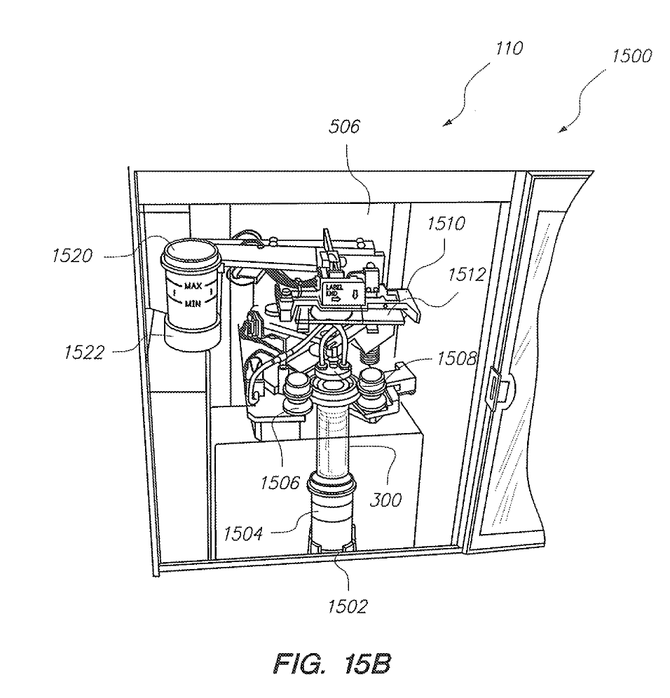

Persons skilled in the art will appreciate that embodiments can be implemented with various components of various slide preparation systems. For reference, FIGS. 15A, 15B, 16 and 17 illustrate how embodiments can be implemented using various slide preparation systems, including the ThinPrep® 2000 (TP2000) and ThinPrep® 3000 (TP3000) machines available from Cytyc Corporation, Marlborough, Mass.

FIGS. 15A and 15B illustrate one system 1500 including components 120 located within the chamber 502 that are used to hold a vial container 1504 and the filter assembly 300. In particular, the slide preparation apparatus 110 includes a vial holder 1502 for holding the vial container 1504, and two bobbins 1506 for engaging with the first portion of the filter assembly cap 320. A specimen sample 1530 that was collected from a patient and placed in a vial 1504, which includes a container 1532 for holding the sample 1530, and a cap 1534 for sealing the sample 1530 within the container 1532.

In some embodiments, the slide preparation apparatus 110 can further include a clamp for releasably engaging the filter assembly 300. The slide preparation apparatus 110 further includes a cap seal 1508 for engaging with the filter assembly cap 320, and a slide handler/ejector 1510 for holding/ejecting the slide 1512. The slide preparation apparatus 110 further includes a pneumatic system (not shown) responsible for generating precisely controlled positive and negative pressures, and electronics (not shown) which power the machine 110 and allow the processor 112 to control the various motors, valves, and sensors in the slide preparation apparatus 110. A fixative vial 1520 held by a holder 1522 includes a fixative solution for preparing a specimen slide.

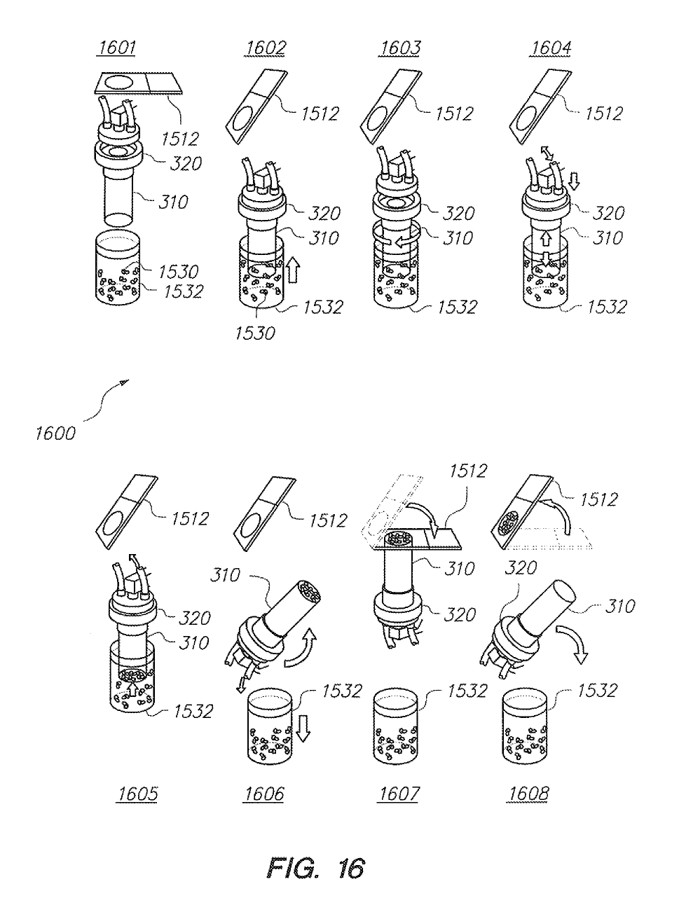

FIG. 16 illustrates a process 1600 that is performed by the slide preparation apparatus 110 in accordance with some embodiments. After the slide 1512, the vial container 1504 holding the sample, and the filter assembly 300, are in place (Step 1601), an elevator (not shown) within the slide preparation apparatus 110 then raises the vial container 1504 so that the filter 310 is in contact with fluid within the vial container 1504 (Step 1602). The filter 310 is then rotated by the bobbins 1506 in the slide preparation apparatus 110 to thereby disperse the sample material, break up blood, mucus, non-diagnostic debris, and thoroughly mix the sample (Step 1603). Next, liquid is drawn into the filter 310, and then pushed out, to thereby wet the interior of the filter 310 (Step 1604). The slide preparation apparatus 110 next creates one or more negative pressure pulses (e.g., using the pneumatic system) to draw the sample material towards the filter element 310 in a controlled manner, thereby collecting a thin, even layer of diagnostic cellular material at the filter element 310 (Step 1605).

After a sufficient quantity of sample material has been collected by the filter 310, the filter 310 is inverted, and waste is cleared to a waste bottle (which may be the vial container 150) (Step 1606). The turning of the filter 310 can be accomplished by using a positioner (not shown) located within the slide preparation apparatus 110. Next, the slide 1512 is brought into contact with the filter element 310, thereby transferring sample cells to the slide 1512 (Step 1607). In some embodiments, the cellular material can be transferred to the glass slide 1512 using computer controlled mechanical positioning and positive air pressure (which “pushes” the material towards the slide 1512). The positive air pressure may be created by the pneumatic system (not shown) within the slide preparation apparatus 110. In some embodiments, the processor 112 monitors the rate of flow through the filter 310 during the collection process to prevent the collected cellular material at the filter element 310 from being too scant or too dense.

After the sample material has been transferred to the slide 1512, the slide 1512 is then deposited into a fixative bath, and is ejected (Step 1608). In other embodiments, the slide 1512 is ejected into a cell fixative bath, ready for staining and evaluation. In some embodiments, the above process results in a slide 1512 that has a uniform thin-layer, and is free of obscuring artifacts such as blood, mucus and inflammation. The created slide 1512 can then be evaluated by laboratory personnel, as in a diagnostic procedure.

In any of the embodiments described herein, the processor 112 can be configured to process a sample based on the information stored in the information storage device 410. For example, if the information stored in the information storage device 410 includes a filter version, “V1”, then the processor 112 may cause the slide preparation apparatus 110 to draw the sample material towards the filter element 310 for a first duration (e.g., 3 minutes). On the other hand, if the filter version is “V2′, then the processor 112 may cause the slide preparation apparatus 110 to draw the sample material towards the filter element 310 for a second duration (e.g., 6 minutes). As such, the processor 112 can be configured to vary a manner in which the processed is performed based on different types of filters. In other embodiments, the filter version can be represented by a serial number or a code that indicates a type of filter.

In other embodiments, the information storage device 410 stores one or more processing parameters (e.g., an amount of elevation raised by the elevator, a speed and/or a duration of rotation by the bobbins 1506, an amount of liquid drawn into/pushed out of the filter 310, a duration of sample material drawing, an amount and/or duration of positive or negative air pressure created by the pneumatic system, and/or a density or an amount of sample material desired to be collected). In such cases, the processor 112 can be configured to perform the above process (or a similar process) based on the processing parameter(s). As used in this specification, the term “processing parameter” refers to a parameter that can be used to create, define, modify, or eliminate, a process or a subpart (e.g., a step) of the process. In further embodiments, the processor 112 can be configured to calibrate the slide preparation apparatus 110 based on information 412 (e.g., a calibration information) stored in the information storage device 410.

In should be noted that the slide preparation apparatus 110 is not limited to the example described previously, and that the slide preparation apparatus 110 can have other configurations in other embodiments. For example, in other embodiments, instead of processing one sample at a time, the slide preparation apparatus 110 can be configured to process multiple samples in a batch.

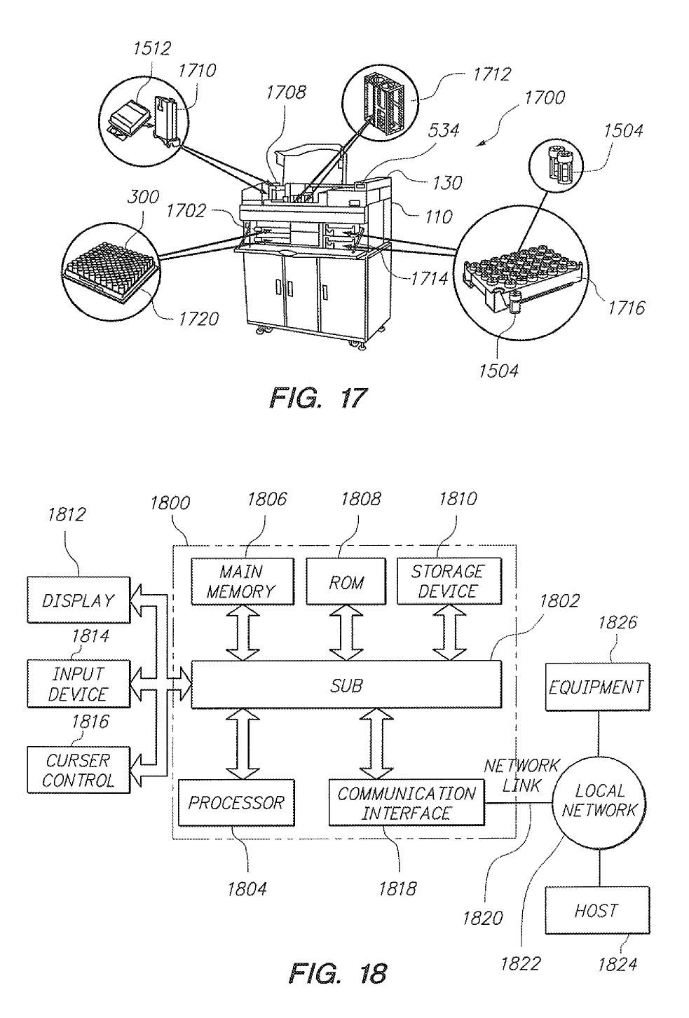

For example, FIG. 17 illustrates a variation of the slide preparation system 1700 in accordance with other embodiments. The slide preparation system 1700 includes a slide preparation apparatus 110, which in the illustrated embodiments, is a ThinPrep® 3000 machine available from Cytyc Corporation, at Marlborough, Mass. The slide preparation apparatus 110 is configured to process multiple samples in a batch. The slide preparation apparatus 110 includes a first chamber 1702 or housing one or more trays 1720 of filter assemblies 300, a cartridge holder 1708 for holding a slide cartridge 1710 of one or more slides 1512, a stacking rack 1712, and a second chamber 1714 for housing one or more trays 1716 of vials 1504. The slide preparation system 1700 also includes a data retrieval device 130 coupled to the slide preparation apparatus 110, as similarly discussed herein.

When using the slide preparation system 1700 of FIG. 17, the vial tray 1716 is first loaded with the vials 1504 (which may or may not be capped), and the loaded tray 1716 is placed into the second chamber 1714. Next, the filter tray 1720 is loaded with the filter assemblies 300, and is placed within the first chamber 1702. The slide cartridge 1710 is then loaded with the slides 1512, and is placed in the cartridge holder 1708. The information storage device 410 is then placed within the slot 534 of the data retrieval device 130, such as a card reader. The data retrieval device 130 receives information 142 from the information storage device 410 regarding the filter assemblies 300, and transmits such information to the processor 112 for processing. The processor 112 then operates the slide preparation apparatus 110 based on the information.

In some embodiments, the information comprises a value indicating a number of sample/filter assemblies 300 that can be processed. In such cases, if the number of filter assemblies 300 exceeds the value 412 stored in the information storage device 410, the processor 112 does not allow the slide preparation apparatus 110 to be activated or operate. Alternatively, if the number of filter assemblies 300 exceeds the value 412 stored in the information storage device 410, the processor 112 can cause the slide preparation apparatus 110 to process the filter assemblies 300 up to the number represented by the value. In some embodiments, if the processor 112 does not receive data from the data retrieval device 130 (e.g., no information storage device 410 is inserted into the slot 534), the processor 112 may be configured to not send an activation signal to thereby inhibit the slide preparation apparatus 110 from being started. The technique of using the filter assemblies 300 to create slides 1512 is similar to that discussed with reference FIG. 16.

As described previously, the processor 112 may be implemented using a computer. For example, one or more instructions can be imported into a computer to enable the computer to perform any of the functions described herein. FIG. 18 is a block diagram that illustrates an embodiment of a computer system 1800 upon which embodiments of the processor 112 may be implemented. Computer system 800 includes a bus 1802 or other communication mechanism for communicating information, and a processor 1804 coupled with bus 1802 for processing information. Computer system 1800 also includes a main memory 1806, such as a random access memory (RAM) or other dynamic storage device, coupled to bus 1802 for storing information and instructions to be executed by processor 1804. Main memory 1806 also may be used for storing temporary variables or other intermediate information during execution of instructions to be executed by processor 1804. Computer system 1800 may further include a read only memory (ROM) 1808 or other static storage device(s) coupled to bus 1802 for storing static information and instructions for processor 1804. A data storage device 1810, such as a magnetic disk or optical disk, is provided and coupled to bus 1802 for storing information and instructions.

Computer system 1800 may be coupled via bus 1802 to a display 1812, such as a cathode ray tube (CRT), for displaying information to a user. An input device 1814, including alphanumeric and other keys, is coupled to bus 1802 for communicating information and command selections to processor 1804. Another type of user input device is cursor control 1816, such as a mouse, a trackball, cursor direction keys, or the like, for communicating direction information and command selections to processor 1804 and for controlling cursor movement on display 1812. This input device typically has two degrees of freedom in two axes, a first axis (e.g., x) and a second axis (e.g., y), that allows the device to specify positions in a plane.

Embodiments described herein are related to the use of computer system 1800 for processing electronic data, and/or controlling an operation of the slide preparation apparatus 110. According to some embodiments, such use may be provided by computer system 1800 in response to processor 1804 executing one or more sequences of one or more instructions contained in the main memory 1806. Such instructions may be read into main memory 1806 from another computer-readable medium, such as storage device 1810. Execution of the sequences of instructions contained in main memory 1806 causes processor 1804 to perform the process steps described in this specification. One or more processors in a multi-processing arrangement may also be employed to execute the sequences of instructions contained in main memory 1806. In alternative embodiments, hard-wired circuitry may be used in place of or in combination with software instructions to implement various operations/functions described herein. Thus, embodiments are not limited to any specific combination of hardware circuitry and software.

The term “computer-readable medium” as used herein refers to any medium that participates in providing instructions to processor 1804 for execution. Such a medium may take many forms, including but not limited to, non-volatile media, volatile media, and transmission media. Non-volatile media includes, for example, optical or magnetic disks, such as storage device 1810. Volatile media includes dynamic memory, such as main memory 1806. Transmission media includes coaxial cables, copper wire and fiber optics, including the wires that comprise bus 1802. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 1804 for execution. For example, the instructions may initially be carried on a magnetic disk of a remote computer. The remote computer can load the instructions into its dynamic memory and send the instructions over a telephone line using a modem. A modem local to computer system 1800 can receive the data on the telephone line and use an infrared transmitter to convert the data to an infrared signal. An infrared detector coupled to bus 1802 can receive the data carried in the infrared signal and place the data on bus 1802. Bus 1802 carries the data to main memory 1806, from which processor 1804 retrieves and executes the instructions. The instructions received by main memory 1806 may optionally be stored on storage device 810 either before or after execution by processor 1804.

Computer system 1800 also includes a communication interface 1818 coupled to bus 1802. Communication interface 818 provides a two-way data communication coupling to a network link 1820 that is connected to a local network 1822. For example, communication interface 1818 may be an integrated services digital network (ISDN) card or a modem to provide a data communication connection to a corresponding type of telephone line. As another example, communication interface 1818 may be a local area network (LAN) card to provide a data communication connection to a compatible LAN. Wireless links may also be implemented. In any such implementation, communication interface 1818 sends and receives electrical, electromagnetic or optical signals that carry data streams representing various types of information.

Network link 1820 typically provides data communication through one or more networks to other devices. For example, network link 1820 may provide a connection through local network 1822 to a host computer 1824. Network link 1820 may also transmits data between an equipment 1826 and communication interface 1818. The data streams transported over the network link 1820 can comprise electrical, electromagnetic or optical signals. The signals through the various networks and the signals on network link 1820 and through communication interface 1818, which carry data to and from computer system 1800, are exemplary forms of carrier waves transporting the information. Computer system 1800 can send messages and receive data, including program code, through the network(s), network link 1820, and communication interface 1818. Although one network link 1820 is shown, in alternative embodiments, communication interface 1818 can provide coupling to a plurality of network links, each of which connected to one or more local networks. In some embodiments, computer system 1800 may receive data from one network, and transmit the data to another network. Computer system 1800 may process and/or modify the data before transmitting it to another network.

Although particular embodiments have been shown and described, it should be understood that the above discussion is not intended to limit the scope of these embodiments. Various changes and modifications may be made without departing from the scope of the claims. For example, persons skilled in the art will appreciate that embodiments can be applied to various components, including filters, filter assemblies, slides, vials and other components and may use various types of storage devices, read devices, write devices and information or data associate with a component. Further, persons skilled in the art will appreciate that embodiments can be used with slide processing systems and other related or similar systems and machines. Thus, embodiments are intended to cover alternatives, modifications, and equivalents that fall within the scope of the claims.

What is claimed is:

1. A system for preparing biological specimen slides, comprising:
a slide preparation apparatus for preparing the specimen slides;
a processor configured to control the slide preparation apparatus; and
a data retrieval device coupled to the processor and configured to retrieve information from a storage device associated with the slide preparation apparatus or a component used by the slide preparation apparatus, wherein the processor selectively enables operation of the slide preparation apparatus based on the retrieved information.

2. The system of claim 1, the processor being a part of the slide preparation apparatus, the data retrieval device being coupled to the processor through a connector of the slide preparation apparatus.

3. The system of claim 1, the data retrieval device being configured to retrieve the information from a storage device attached to the component.

4. The system of claim 1, the data retrieval device comprising a card reader that retrieves the information from a programmable card.

5. The system of claim 1, the data retrieval device comprising a read/write device, the processor being configured to selectively control the read/write device to add to or change the information in the storage device in response to processing respective specimen slides.

6. The system of claim 1, the component comprising a filter assembly used for collecting cells of a biological specimen that are suspended in liquid.

7. The system of claim 1, the system comprising a plurality of slide preparation apparatuses connected to each other through a network, and wherein information relating to a component used by one slide preparation apparatus of the plurality is transmitted other slide preparation apparatus of the plurality over the network.

8. The system of claim 1, wherein the information comprises a number of components that can be used by the slide preparation apparatus.

9. The system of claim 1, wherein the information comprises a number of specimen slides that is authorized to be prepared by the slide preparation apparatus.

10. The system of claim 1, wherein the information includes encrypted data and a unique identification number associated with the component.

11. A method of preparing a specimen slide having cells of a biological specimen using a slide preparation apparatus controlled by a processor coupled to a data retrieval device, the data retrieval device configured to retrieve information from a storage device associated with the slide preparation apparatus or a component used by the slide preparation apparatus, the method comprising:
retrieving information associated with a slide preparation apparatus or a component used by the slide preparation apparatus to prepare a specimen slide; and
selectively operating the slide preparation apparatus based on the retrieved information.

12. The method of claim 11, wherein the data retrieval device retrieves the information from a storage device attached to the component.

13. The method of claim 11, the data retrieval device comprising a card reader that retrieves the information from a programmable card.

14. The method of claim 11, the data retrieval device comprising a read/write device, the method further comprising selectively adding to or changing the information in the storage device in response to processing respective specimen slides.

15. The method of claim 11, the component comprising a filter assembly used for collecting cells of a biological specimen that are suspended in liquid.

16. The method of claim 11, further comprising transmitting information relating to a component used by one slide preparation apparatus to another slide preparation apparatus.

17. The method of claim 11, wherein the information is comprises one or more of a number of components that can be used by the slide preparation apparatus and a number of specimen slides that is authorized to be prepared by the slide preparation apparatus.

18. A specimen preparation kit for use in preparing a biological specimen slide, comprising:
a filter assembly used for collecting biological specimen cells suspended in a liquid;
a storage device associated with the filter assembly; and
a processor that selectively enables operation of a slide preparation apparatus to use the filter assembly to prepare a specimen slide based on information stored on, retrieved from, the storage device.

19. The specimen preparation kit of claim 18, the storage device comprising a card.

20. The specimen preparation kit of claim 18, wherein the storage device is attached to the filter assembly.

21. The specimen preparation kit of claim 18, further comprising a tray for holding the filter assembly and the storage device.
